(19) United States
(12) Patent Application Publication
(10) Pub. No.: US 2023/0027816 A1
(43) Pub. Date: Jan. 26, 2023

(54) OBLIQUE DIAPHYSEAL OSTEOTOMY SYSTEM FOR METATARSAL SHORTENING

(71) Applicant: Inverted Ortho, LLC, Cincinnati, OH (US)

(72) Inventor: Robert Kulwin, Cincinnati, OH (US)

(21) Appl. No.: 17/871,895

(22) Filed: Jul. 22, 2022

Related U.S. Application Data
(60) Provisional application No. 63/224,717, filed on Jul. 22, 2021.

(51) Int. Cl. A61B 17/15; A61B 17/80

(52) U.S. Cl. CPC A61B 17/151; A61B 17/8061; A61B 17/1775

(57) ABSTRACT

An oblique metatarsal shortening osteotomy system comprising a cut guide, wherein the cut guide comprises one or more pre-drilled fixation holes, wherein the cut guide can be placed on a metatarsal bone to enable an oblique shortening osteotomy of varying lengths, and wherein the pre-drilled fixation holes enable fixation of the cut guide to the metatarsal bone. The cut guide may comprise a central block. The cut guide may comprise one or more fixation flanges. The cut guide may comprise one or more slots to enable cuts to be made in a metatarsal. The cut guide may enable metatarsal compression through pre-drilling without need for alternative techniques. Angulation of the pre-drilling may allow for fixation to allow for intramedullary fixation to be placed within a medullary canal of an osteotomized metatarsal.

### TECHNICAL FIELD

Embodiments of the technology relate, in general, to surgical cut guide technology, and in particular, to a cutting guide system that allows for measured resection of a metatarsal bone in order to shorten the bone. Specifically, the process allows for a reproducible shortening without inducing angulation, and for an oblique diaphyseal osteotomy for improved union rates compared to the current standard procedure. Furthermore, the cutting guide design allows for compatibility with a plating system to prevent the need for re-drilling to apply plate fixation as well as set alignment for the reduction to the plate.

### BACKGROUND

Metatarsal shortening osteotomies are largely achieved via two methods: a transverse diaphyseal shortening osteotomy or an oblique intraarticular shortening osteotomy about the metatarsophalangeal joint. The transverse shortening method spares the joint, but has frequent problems with non-unions due to the diaphyseal and transverse nature of the osteotomy. The oblique intraarticular shortening osteotomy (also referred to as a “Weil” osteotomy) has good union rates, but is technically challenging and can result in avascular necrosis of the metatarsal head, osteoarthritis, or plantar translation of the metatarsal head, which results in persistent pain. There is a need for an osteotomy system that avoids the complications of both.

Currently available solutions include a system that makes an oblique osteotomy through a specialized plate, as well as an external jig for measured resection for Weil osteotomies. The measured resection for Weil osteotomies still has the same drawbacks as a traditional Weil. The plate that allows for an oblique osteotomy has not been widely adopted for a number of reasons: the plate is rather large and bulky, the osteotomy is oblique but does not resect bone, rather relies on translation to shorten the bone. This does shorten the bone, but also translates the metatarsal laterally. Furthermore, it limits the amount of shortening due to the design. There is a need for a cut guide that allows for a stepwise procedure that eliminates the many variables present in freehand resections.

It is well described that oblique osteotomies in other areas of the body (specifically, the ulna for ulnar shortening osteotomies) have increased union rates compared to transverse. However, ulnar osteotomies are based on a plate, and therefore, an expensive implant. The system used for ulnar shortening osteotomies would not translate to metatarsal bone because of anatomic constraints on metatarsals. There is a desire for pre-planned measured resection that does not result in translation of the metatarsal, only longitudinal shortening. There is a need for low-profile plating to reduce risk of need for removal of implants in a second surgery. Lastly, there is a desire to avoid the complications of Weil osteotomies. Outcomes for the surgical treatment of arthritis or avascular necrosis of lesser metatarsophalangeal joints are quite poor, so there is a need to avoid a potentially unsalvageable complication.

### BRIEF DESCRIPTION OF THE DRAWINGS

The drawings and detailed description that follow are intended to be merely illustrative and are not intended to limit the scope of the invention as contemplated by the inventor. The detailed description of specific embodiments herein can be best understood when read in conjunction with the following drawings.

FIG. 1 shows an exemplary osteotomy system that involves applying a first embodiment of a cut guide to a designated metatarsal.

FIG. 2 is a perspective view of a second embodiment of a cut guide;

FIG. 3 is a top view of the cut guide shown in FIG. 2;

FIG. 4 is a side view of the cut guide shown in FIG. 2;

FIG. 5 is a perspective view of a third embodiment of a cut guide;

FIG. 6 is a top view of the cut guide shown in FIG. 5;

FIG. 7 is a side view of the cut guide shown in FIG. 5;

FIG. 8 is a perspective view of a fourth embodiment of a cut guide;

FIG. 9 is a top view of the cut guide shown in FIG. 8;

FIG. 10 is a side view of the cut guide shown in FIG. 8;

FIG. 11 is a perspective view of a fifth embodiment of a cut guide;

FIG. 12 is a rotated perspective view of the cut guide shown in FIG. 11;

FIG. 13 is a top view of the cut guide shown in FIGS. 11 and 12;

FIG. 14 is a side view of the cut guide shown in FIGS. 11 and 12;

FIG. 15 is a cross-sectional view of a cut guide fixated to a metatarsal bone;

FIG. 16 is a cross-sectional view of a bone saw or burr getting ready to use the cut guide of FIG. 15;

FIG. 17 is a cross-sectional view of a bone saw or burr making a first cut while employing the cut guide of FIG. 15;

FIG. 18 is a cross-sectional view of a bone saw or burr making a second cut while employing the cut guide of FIG. 15;

FIG. 19 is a cross-sectional view of the cut guide still in place on a metatarsal after the resected bone is removed;

FIG. 20 is a cross-sectional view of the joined metatarsal bones and a plating system;

FIG. 21 is a medial side view of a sixth embodiment of a cut guide;

FIG. 22 is a top view of the cut guide shown in FIG. 21;

FIG. 23 is a medial perspective view of the cut guide shown in FIG. 21;

FIG. 24 is a medial side view of a seventh embodiment of a cut guide;

FIG. 25 is a top view of the cut guide shown in FIG. 24; and

FIG. 26 is a medial perspective view of the cut guide shown in FIG. 24.

### DETAILED DESCRIPTION

Various non-limiting embodiments of the present disclosure will now be described to provide an overall understanding of the principles of the structure, function, and use of the compositions, methods, and processes disclosed herein. One or more examples of these non-limiting embodiments are illustrated in the accompanying drawings. Those of ordinary skill in the art will understand that compositions and methods specifically described herein are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

The inventor conceived of a novel cut guide system that, for the purpose of illustration, is disclosed herein as applied in the context of an oblique diaphyseal osteotomy system for metatarsal bone (or simply “metatarsal”) shortening. While the disclosed applications of the inventor's osteotomy system satisfy a long-felt but unmet need in the art of osteotomies, it should be understood that the inventor's osteotomy system is not limited to being implemented in the precise manners set forth herein, but could be implemented in other manners without undue experimentation by those of ordinary skill in the art in light of this disclosure. Accordingly, the examples set forth herein should be understood as being illustrative only, and should not be treated as limiting.

Generally, the system described herein allows for reproducible measured resection, without risk to the metatarsophalangeal joints, with significantly reduced rates of nonunion. It allows for use of standard, low-profile plating that reduces re-operation risk and keeps implant costs down. The present osteotomy system avoids the complications of traditional methods: it spares the joint, while providing a larger surface area of bone for healing and thus reducing the rate of non-union. This removable guide for an oblique shortening osteotomy allows for perfect alignment of the plate for the plane of the osteotomy. In some embodiments, the cut guide allows for the guide placement to be reused for plate fixation.

The cut guide can be made using any process, such as injection molding or 3D printing. The cut guide may be constructed from a number of different materials. For instance, one or more polymers (e.g., a polyamide polymer like nylon), stainless steel, titanium alloy, cobalt chrome, or any of many metallic alloys approved for use in orthopedic cutting devices, or combinations thereof. The cut guide can be patient-specific or generalized. The cut guide can be a one-time-use product or it may be reusable. The cut guide may comprise a handle.

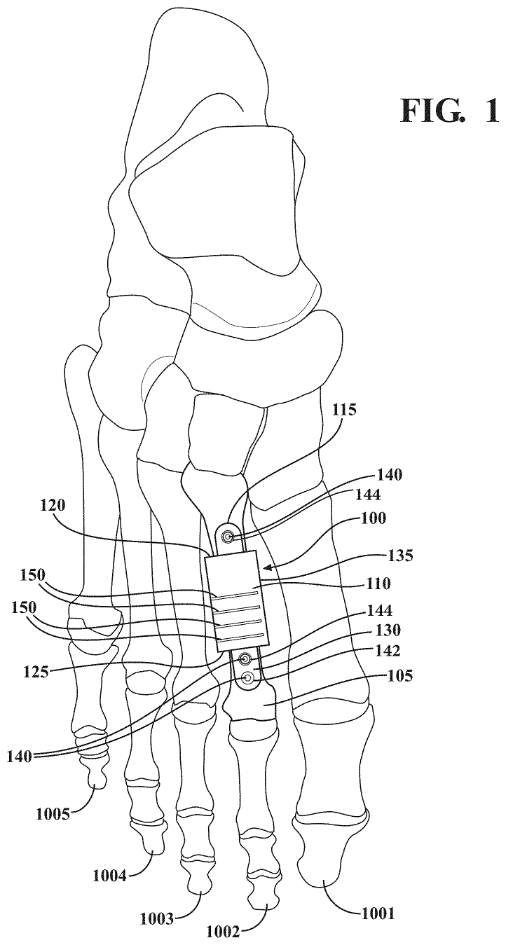

FIG. 1 shows a first embodiment of an exemplary osteotomy system that involves applying a cut guide 100 to a designated metatarsal bone 105. The cut guide allows for oblique, measured resection of predetermined length of resection. The cut guide 100 may be used to resection a first metatarsal 1001, a second metatarsal 1002, a third metatarsal 1003, a fourth metatarsal 1004, or a fifth metatarsal 1005. A bone saw or a burr may be used with the cut guide for resection. A burr can allow for a lower profile and narrower cut guide.

In some embodiments, the cut guide 100 comprises a central block 110 between one or more fixation flanges 115 on the ends of the central block to help center the cut guide on the bone 105. In some embodiments, the central block 110 is generally wider than the fixation flanges 115. In some embodiments, the cut guide 100 comprises two fixation flanges: one fixation flange 115 is located at the proximal (ankle) end 120 of the central block 110 and the other fixation flange 130 is located at the distal (toe) end 125 of the central block 110. In other embodiments, there may be more than two flanges, for instance, in addition to fixation flanges, positioning flanges may protrude off the medial side or lateral side 135 proximally and distally that go deep to the bone and help align the guide. The fixation flanges 115, 130 each comprise one or more holes 140 that are congruent with a plating system. The central block 110 comprises one or more slots 150. The central block 110 may be open on one side to allow for a wider range of saw blade use. Furthermore, the proximal flange 115 or distal flange 130 may only have one hole if preferred. It is possible that a fixation hole need be incorporated into the central block 110 based on the fixation method used.

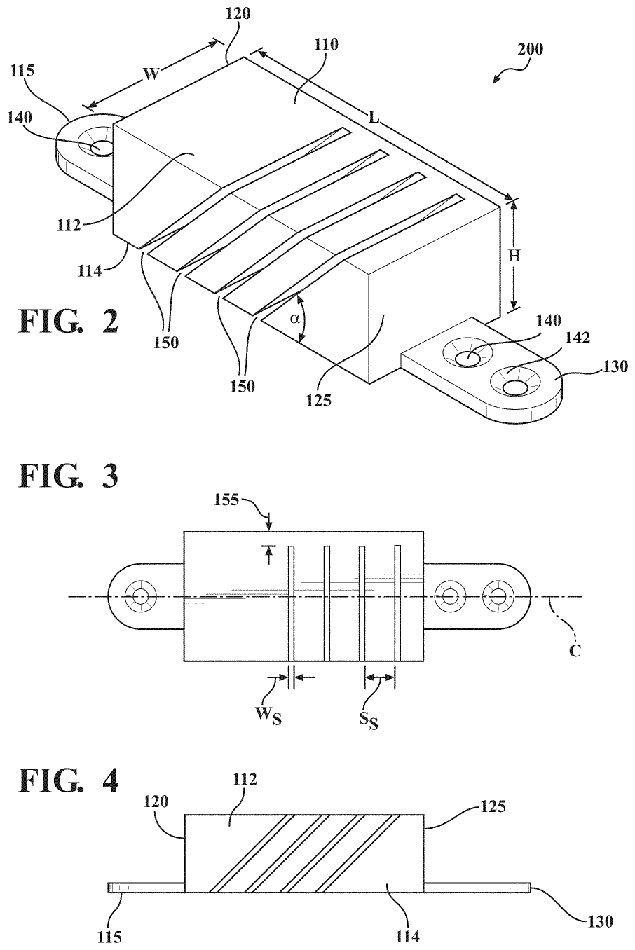

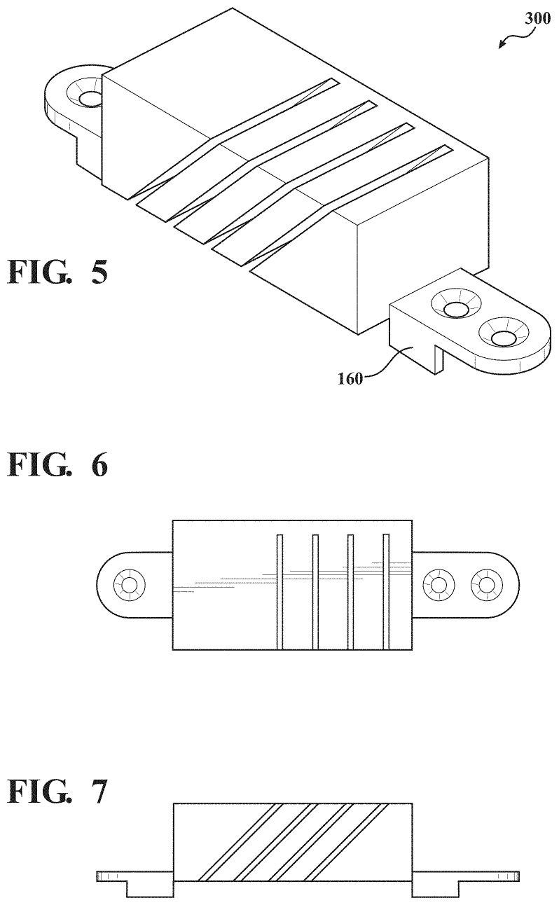

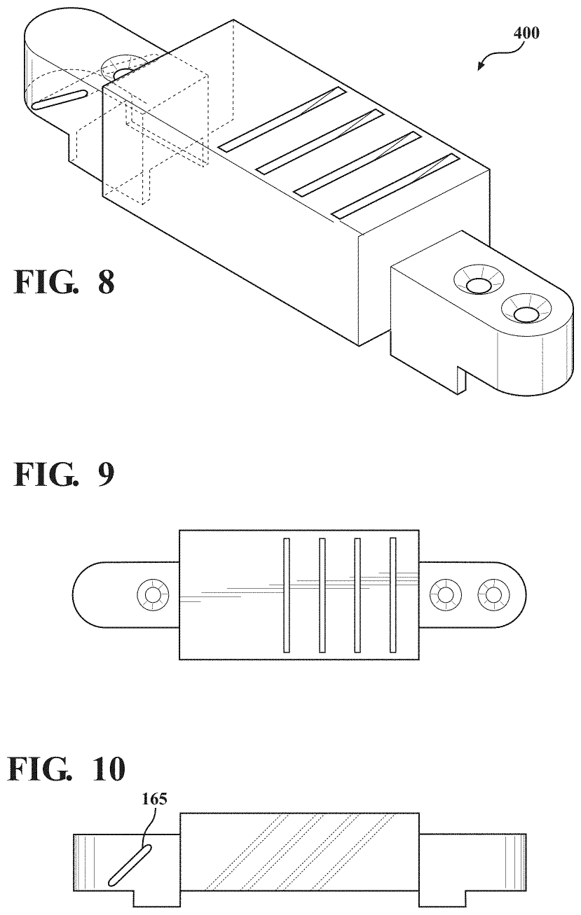

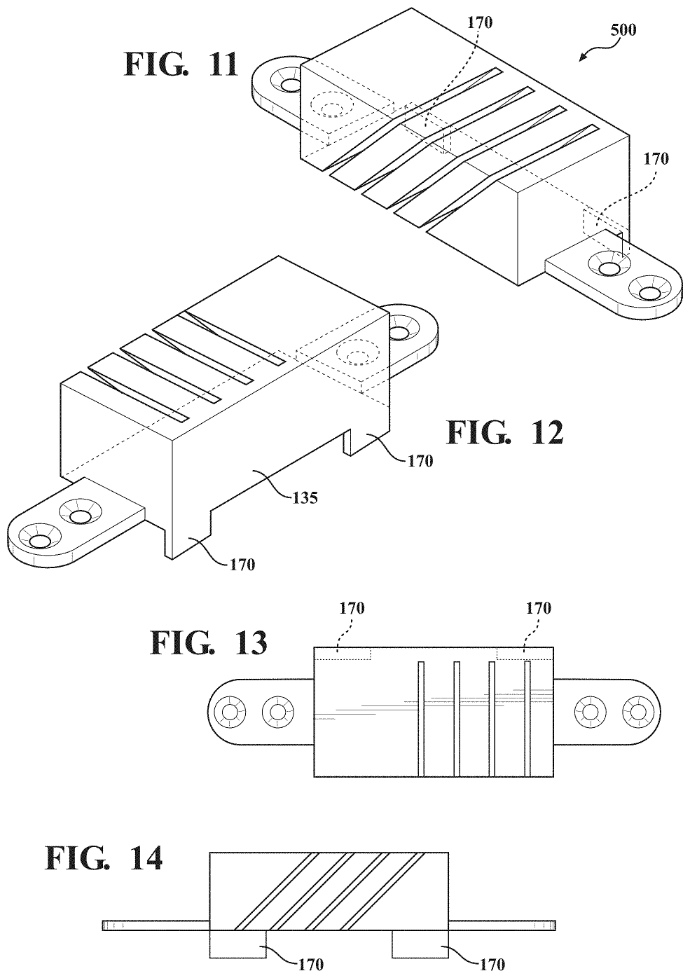

The flanges may be designed to complement the specific anatomy or equipment used in a procedure. Multiple exemplary embodiments of cut guides are illustrated herein: a first embodiment (FIG. 1), a second embodiment (FIGS. 2-4), a third embodiment (FIGS. 5-7), a fourth embodiment (FIGS. 8-10), and a fifth embodiment (FIGS. 11-14). Note the varying flange designs in the side views of FIGS. 4, 7, 10, and 14.

The fixation flanges each comprise one or more holes that are congruent with a plating system. In some embodiments, one or more holes may comprise a receiving lip around the hole that is higher than or lower than the flange top surface to aid in guiding the fixation wire. For instance, as shown in FIG. 1, two holes comprise a raised receiving lip 144 around the holes and one hole comprises a lowered receiving lip 142 around the hole. In some embodiments, the hole diameter is about 1 mm to about 10 mm. In some embodiments, the hole diameters are all the same (see, e.g., FIG. 3). In other embodiments, the hole diameters are all different, or promixal end holes have a diameter different from distal end holes (see, e.g., FIG. 1), or at least one hole is different from the rest. In the fifth embodiment, shown in FIGS. 11-14, the cut guide comprises two holes for points of fixation at the distal (toe side) of the metatarsal, and two holes for the proximal (ankle side).

The fixation flange at the distal portion of the cut guide comprises one or more holes designed for fixation, such as olive wire fixation. The fixation flange at the proximal portion of the cut guide comprises one or more holes (which may be slightly offset to the center of the screw hole in the complementary plating system used to allow for compression). In some embodiments, the holes distally are spaced to allow for reuse of these holes for plate fixation after an osteotomy, and are fixated using olive wires of a diameter and length such that the holes do not need to be re-drilled. In the fifth embodiment this will be the case for both the proximal and distal holes, the proximal holes being slightly offset from the corresponding plate to allow for compression through the plate. The cut guide is designed such that a plate will fit right on after the resection has been completed without the need for re-drilling, etc. The design of the olive wires does not require an extra step of drilling the holes distally for plate fixation. Lastly, the distal holes may be divergent up to 20 degrees to the medial and lateral, to allow for wire fixation across the metatarsophalangeal joint and into the shaft of the metatarsal in instances where that is deemed appropriate.

The dimensions of the central block vary depending on the width, kerf, and length of the saw or burr. In some embodiments, for instance as shown in FIG. 2, the central block height H is from about 5 mm to about 40 mm, or about 10 mm. In some embodiments, the central block width W is about 10 mm. In some embodiments, the central block length L is from about 10 mm to about 60 mm, or about 20 mm.

As shown in FIG. 2, in some embodiments, there are multiple oblique slots 150 in the central block 110 allowing for resection of various length segments of bone. Rather than running perpendicular from the top side 112 of the central block 110 to the bottom side 114 of the central block 110, the slots 150 run oblique from the top side 112 to the bottom side 114 of the block 110. The slot angulation from the top side of the block to the bottom side of the block is from distal to proximal. This is an easier angle to work with than reverse obliquity, as the ankle will not obstruct the cutting angle or the hole portion of the cut guide 200. The angle of resection a can vary widely, from about 15 to about 60 degrees, so the slots 150 can be angled appropriately. In some embodiments, the slots 150 are at about a 45-degree angle of resection α. In some embodiments, the slots 150 are at about a 30-degree angle of resection α.

The central block 110 may comprise one to ten slots 150, more preferably two to five slots 150. In some embodiments, the central block comprises three slots 150. In some embodiments, the central block comprises four slots 150.

As shown in FIG. 3, the width of the slots Wₛ may range from about 0.5 mm to about 2 mm. In some embodiments, the width Wₛ is up to about 0.8 mm, up to about 1 mm, or up to about 1.5 mm. The slots 150 may be spaced Sₛ from about 1 mm to about 10 mm apart. In some embodiments, the slot spacing Sₛ is from about 2 mm to about 5 mm. In some embodiments, the slot spacing Sₛ is about 3.6 mm. The cut guide 200 has a centerline C. In some embodiments, the slot margin 155 is less than about 25%, or less than about 10% the width W of the central block 110. Slots 150 in the block 110 may be closed on both the medial and lateral sides—meaning there is a slot margin along the length of each side of the block—or open on the medial (aka small-toe) side or lateral side to allow for saw blade oscillation. FIG. 3 shows a cut guide 200 having a slot margin 155 (i.e., closed) on the lateral side and no slot margin (i.e. open) on the medial side.

In some embodiments, the cut guide length is from about 10 mm to about 100 mm, or about 45 mm. In some embodiments, the cut guide width W is about 10 mm. In some embodiments, the height and/or width of the cut guide varies along its length.

In an embodiment where there are four holes in a cut guide positioned from left to right (e.g., distal end, hole 1, hole 2, hole 3, hole 4, proximal end), hole 1 may be on a distal fixation flange, hole 2 may be between two slots on a central block, hole 3 may be on a proximal fixation flange, and hole 4 may be on the proximal fixation flange. In some embodiments, hole 3 comprises an about 1.87 mm diameter hole clearance to accommodate about 1.57 mm diameter olive wire. In some embodiments, hole 4 comprises an about 2 mm diameter hole clearance to accommodate about 1.6 mm diameter K-wires (Kirschner wires). In one embodiment, the distance from hole 3 to the proximal end is about 17.6 mm. In another embodiment, the distance from hole 3 to the proximal end is about 15.6 mm.

As shown in FIG. 4, the cut guide 200 has a top surface 112, a bottom surface 114, a proximal end 120, and a distal end 125. Fixation flanges 115, 130 may be the same or different lengths, the same or different widths, and may comprise the same number and shape holes or a different number or shape holes. The third embodiment (FIGS. 5-7), fourth embodiment (FIGS. 8-10), and fifth embodiment (FIGS. 11-14) of the cutting guide show varying fixation flange 115, 130 designs showing positioning flanges 160 and a flange slot 165.

In certain embodiments, cut guide design variation allows for positioning flanges that can be narrowed on to the bone medially and laterally to “auto-center” the cut guide. For example, as shown in FIGS. 11-14, positioning flanges 170 may be added to the lateral side 135 of the central block 110 proximally and distally to keep the guide 500 from sliding too medial: because metatarsal bones 105 are quite narrow, it may be most efficient to “center” the cut guide 500 by limiting how far medially the guide 500 can be placed.

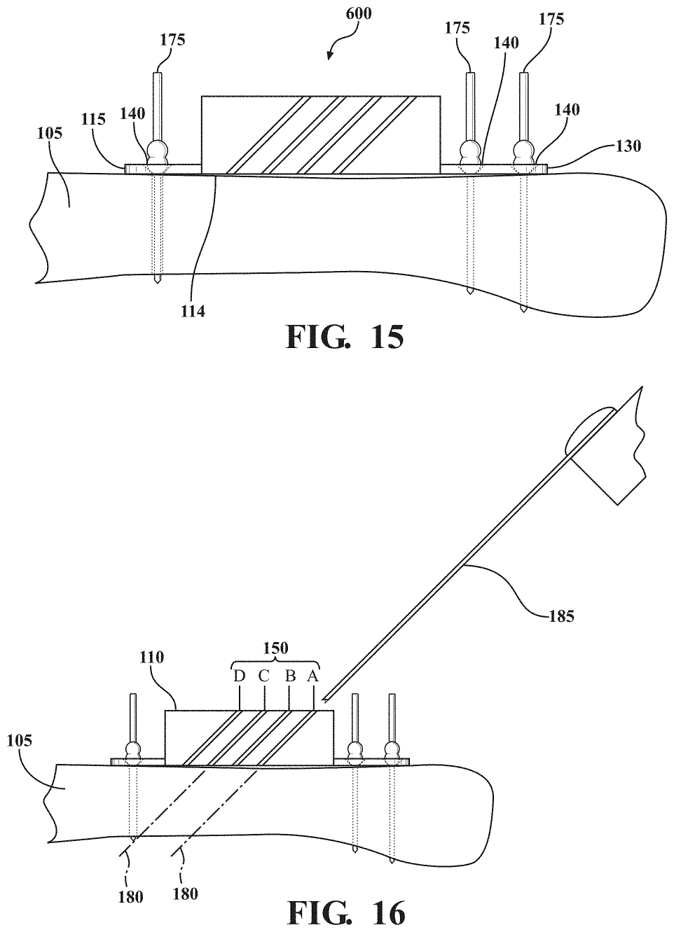

One method for use of an oblique diaphyseal osteotomy system for metatarsal shortening is illustrated in cross-sectional FIGS. 15-20. First, via surgical approach, expose the targeted metatarsal bone 105. Align the centerline of the cut guide 600 along the length of the bone 105. Place the bottom surface 114 of the cut guide 600 along the length of the bone 105 and determine preferred positioning. If present on the central block 110 or fixation flanges 115, 130, positioning flanges 160, 170 that protrude toward the plantar (bottom) surface of the bone 105 can determine medial to lateral placement. Fixate the cut guide 600 proximally and distally. In some embodiments, fixate with olive wires 175 in each fixation flange 115, 130 hole 140. In other embodiments, wire may be used to fixate the proximal fixation flange into the operative metatarsal; for instance, wire fixation from the proximal fixation flange into an adjacent metatarsal may be desired. Ensure that the cut guide centerline is centered on the bone along its length.

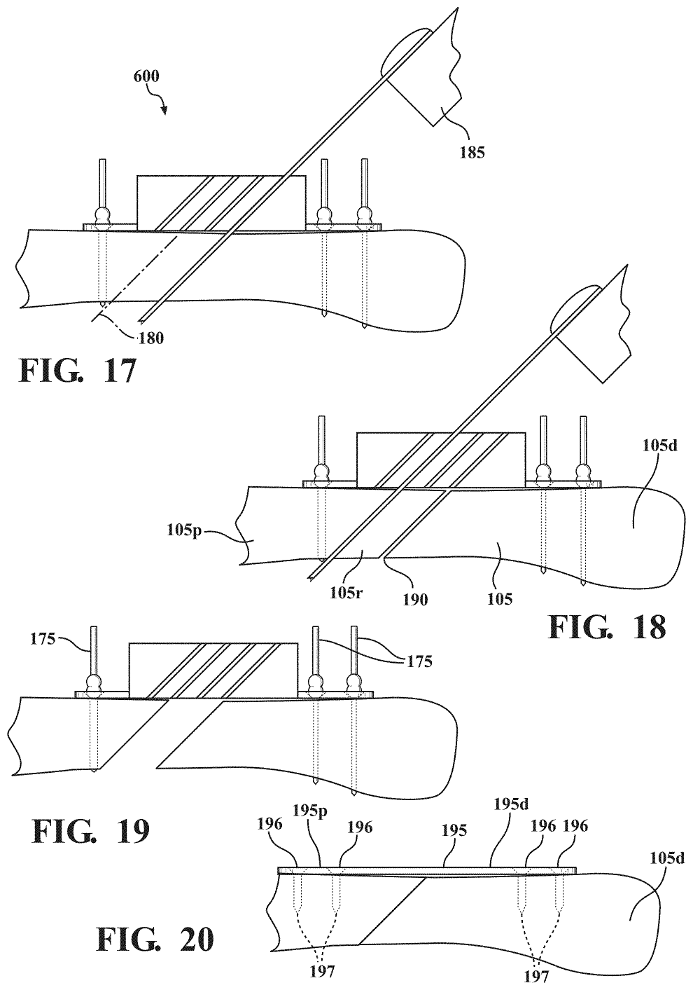

Turning to FIG. 16, once fixated to the bone 105, two slots 150 on the central block 110 of appropriate distance are chosen, for example, slot A and slot C. Desired resection can then be chosen in designs with multiple oblique slots 150 at various spacing Sₛ. As shown in FIGS. 17 and 18, using the cut guide 600 to define the trajectory 180 of a saw or burr 185, parallel oblique cuts 190 are then made through the bone 105. Depending on the desired resection length, resection can be achieved by using multiple slots 150 in the same cut guide (for instance, slot A and slot C, as shown in FIGS. 18 and 19), or slots in two separate cut guides designated for specific resection lengths (e.g., slot A in a first cut guide and slot A in a second cut guide). In some embodiments, the cut guide 600 allows a user to cut about 75% of the length of each trajectory 180 and then complete each cut 190. This enables cuts that are more precise. This yields three sections of bone: a proximal bone 105p, a resected bone 105r, and a distal bone 105d. As shown in FIG. 19, the resected bone 105r is removed first and then the fixation 175 of the cut guide 600 is removed. In some embodiments, after the resection is performed, the fixation 175 of the cut guide 600 is removed first, then the cut guide 600 is removed, then the resected bone 105r is removed.

As shown in FIG. 20, a plate 195 having holes 196 is then placed on the metatarsal bone 105 and attached through the pre-drilled holes. The pre-drilled holes (preferably via olive wire) ensure that the plating occurs precisely in line with the osteotomy both longitudinally and rotationally. To do this, the distal bone 105d is reduced under the distal portion of the plate 195d and fixated with screws 197 placed in the two pre-drilled distal holes 196. The proximal bone 105p is then reduced into an “armpit” created by the angled resection of the distal bone 105d. A small clamp may be used to center the plate on the proximal metatarsal bone. A screw 197 is then placed through a hole 196 in the proximal end of the plate 195p into the pre-drilled hole in the proximal bone 105p and compressed. The final result is a compressed, oblique, diaphyseal osteotomy with maintenance of longitudinal alignment.

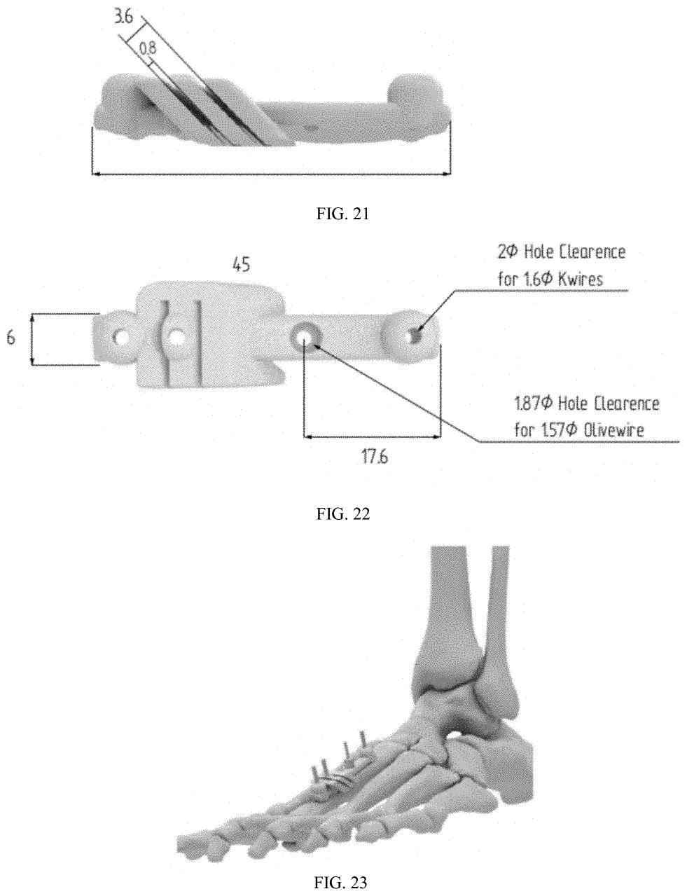

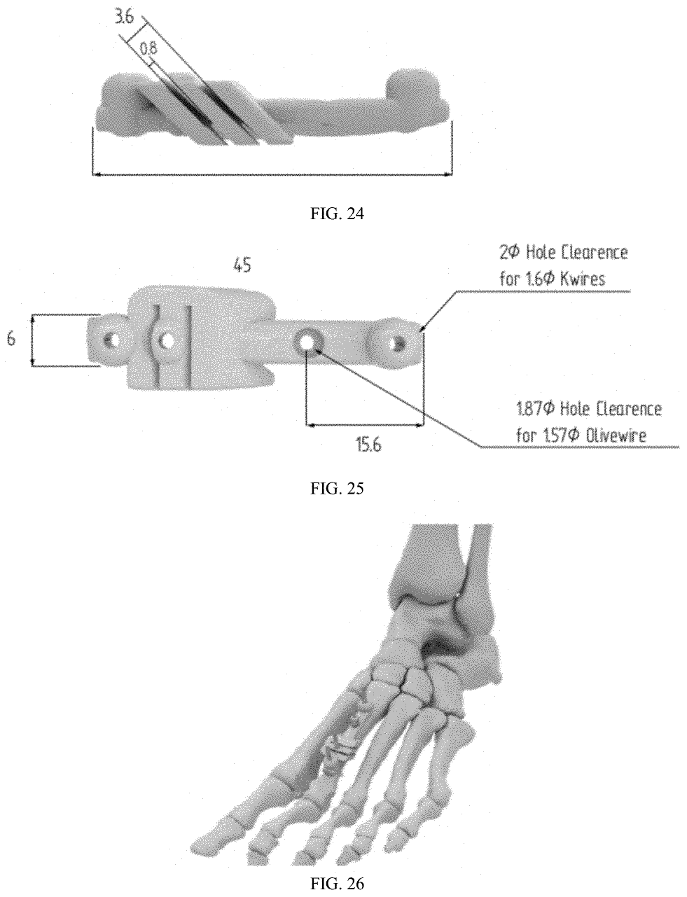

In some embodiments, the plate 195 may have more or less holes 196 than the cut guide. For example, FIG. 20 shows a plate 195 with two holes 196 in its proximal end 195p while the cut guide 600 shown in FIGS. 15-19 only has one hole 140 in its proximal fixation flange 115. In some embodiments, the cut guide and the plate have the same number of holes on each fixation flange/side, such as would be the case if the cut guide shown in FIGS. 11-14 is used in the method for use of the osteotomy system shown in FIGS. 15-20. FIGS. 21-23 and 24-26 show additional cut guide embodiments.

By design, in some embodiments, proximal plate holes may be slightly offset from the proximal fixation flange/proximal pre-drilled holes such that metatarsal compression occurs when the screws are tightened down. Finally, wound closure and fluoroscopic assessment are performed per surgeon preference, completing the procedure.

The plate can vary based on the dimensions of the cut guide. The cut guide can vary depending on desired plate for fixation. The plate can be hybrid for locking and non-locking screw options. The plate can be stainless steel or a titanium alloy, or other material based on surgeon preference. The plate can include positioning flanges to center the proximal bone into the reduction armpit.

In some embodiments, the olive wires comprise longer and thicker threaded stems, and shorter drive wires so as not to block the saw angle for cutting. Alternatively, longer drive wires can be used and cut. In some embodiments, the cut guide can be designed to fit existing plating systems or new kits may be created which comprise complementary cut guides and plating systems.

While specific embodiments were illustrated and described herein, variations and modifications may be made by those skilled in the art without departing from the scope of this disclosure. The present disclosure is for purposes of illustration and not of limitation; it may take many forms other than those explicitly disclosed herein. As such, the claims below shall be read to include all obvious variations and modifications that may be within the spirit of this disclosure.

What is claimed is:

1. An oblique metatarsal shortening osteotomy system comprising a cut guide, wherein the cut guide comprises one or more pre-drilled fixation holes, wherein the cut guide can be placed on a metatarsal bone to enable an oblique shortening osteotomy of varying lengths, and wherein the pre-drilled fixation holes enable fixation of the cut guide to the metatarsal bone.

2. The oblique metatarsal shortening osteotomy system of claim 1, wherein the cut guide comprises one or more slots to enable cuts to be made in a metatarsal.

3. The oblique metatarsal shortening osteotomy system of claim 1, wherein the cut guide enables metatarsal compression through pre-drilling without need for alternative techniques.

4. The oblique metatarsal shortening osteotomy system of claim 3, wherein an angulation of the pre-drilling allows for fixation to allow for intramedullary fixation to be placed within a medullary canal of an osteotomized metatarsal.

5. An oblique metatarsal shortening osteotomy cut guide, comprising:
a. a central block comprising one or more slots;
b. one or more fixation flanges; and
c. one or more pre-drilled fixation holes.

6. The oblique metatarsal shortening osteotomy cut guide of claim 5, wherein the central block is wider than the one or more fixation flanges.

7. The oblique metatarsal shortening osteotomy cut guide of claim 5, wherein the central block comprises at least two slots which are parallel to one another.

8. The oblique metatarsal shortening osteotomy cut guide of claim 5, wherein the guide comprises nylon.

9. The oblique metatarsal shortening osteotomy cut guide of claim 5, wherein the guide comprises four pre-drilled fixation holes.

10. The oblique metatarsal shortening osteotomy cut guide of claim 5, wherein the central block comprises a proximal end and a distal end, and wherein the guide comprises a first fixation flange located at the proximal end of the central block and a second fixation flange located at the distal end of the central block.

11. The oblique metatarsal shortening osteotomy cut guide of claim 5, wherein the one or more fixation flanges comprises one or more pre-drilled fixation holes.

12. The oblique metatarsal shortening osteotomy cut guide of claim 5, wherein the one or more pre-drilled fixation holes are congruent with a plating system.

13. The oblique metatarsal shortening osteotomy cut guide of claim 5, wherein the central block comprises one or more pre-drilled fixation holes.

14. The oblique metatarsal shortening osteotomy cut guide of claim 5, wherein the central block is open on one side.

15. The oblique metatarsal shortening osteotomy cut guide of claim 5, wherein the central block has a length and at least two sides and comprises a slot margin along the length of at least one side.

16. The oblique metatarsal shortening osteotomy cut guide of claim 5, wherein one or more pre-drilled fixation holes comprise a receiving lip around the hole.

17. The oblique metatarsal shortening osteotomy cut guide of claim 5, wherein the fixation flange at the proximal end of the cut guide comprises one or more holes which are slightly offset to the center of a screw hole in a complementary plate.

18. The oblique metatarsal shortening osteotomy cut guide of claim 5, wherein the slots run oblique from a top side of the block to a bottom side of the block.

19. The oblique metatarsal shortening osteotomy cut guide of claim 5, wherein the slot angulation from the top side of the block to the bottom side of the block is from distal to proximal.

20. The oblique metatarsal shortening osteotomy cut guide of claim 5, wherein the slots are at an angle of about 45 degrees.
